(19) United States
(12) Patent Application Publication
(10) Pub. No.: US 2009/0076447 A1
(43) Pub. Date: Mar. 19, 2009

(54) Method and apparatus for conducting peripheral vascular disease procedures using a novel anchor balloon catheter

(76) Inventors: Jack Casas, (US); David Camacho, (US)

Correspondence Address: RYLANDER & ASSOCIATES PC, 406 West 12th Street, Vancouver, WA, 98660, US

(21) Appl. No.: 11/903,062

(22) Filed: Sep. 19, 2007

(51) Int. Cl. A61M 25/10; A61M 29/02

(52) U.S. Cl. 604/96.01; 606/194

(57) ABSTRACT

An anchor balloon for conducting peripheral vascular disease procedures in an remote entry point such as an opposite extremity is provided, and includes a flexible catheter, which includes a wire lumen and a balloon lumen with a control port for connecting to balloon control and a balloon inflation port; and, a compliant anchor balloon connected near the flexible catheter distal end. A method for treating peripheral vascular disease in an opposite femoral artery includes inserting a first flexible catheter with flexible guide wire into an artery on an opposite side, advancing the flexible catheter to a location upstream of the surgery location, advancing the guide wire through the catheter to an anchor location, withdrawing the first catheter leaving the wire in place, inserting an anchor balloon catheter over the wire to an anchor location, withdrawing the first wire; inflating the anchor balloon against the arterial walls, inserting a stiff guide wire through the anchor balloon catheter to the point of surgery, deflating the anchor balloon, and withdrawing the anchor balloon catheter while leaving the stiff wire in place for further procedures.

### FIELD OF THE INVENTION

The present invention relates to catheters and catheter based surgical operations. More particularly, the present invention relates to catheters and procedures for peripheral artery disease surgery.

### BACKGROUND

In medicine, peripheral artery occlusive disease, also known as peripheral vascular disease (PVD) and peripheral artery disease (PAD), refers to diseases caused by the obstruction of large peripheral arteries, which can result from atherosclerosis, inflammatory processes leading to stenosis, an embolism or thrombus formation. It causes either acute or chronic ischemia.

Mild PAD may be asymptomatic or cause intermittent claudication; severe PAD may cause rest pain with skin atrophy, hair loss, cyanosis, ischemic ulcers, and gangrene. Diagnosis is by history, physical examination, and measurement of the ankle-brachial index. Treatment of mild PAD includes risk factor modification, exercise, antiplatelet drugs, and cilostazol or possibly pentoxifylline as needed for symptoms. Severe PAD usually requires angioplasty or surgical bypass and may require amputation. Prognosis is generally good with treatment, although mortality rate is relatively high because coronary artery or cerebrovascular disease often coexists.

Typically, PAD causes intermittent claudication, which is a painful, aching, cramping, uncomfortable, or tired feeling in the legs that occurs during walking and is relieved by rest. Claudication usually occurs in the calves but can occur in the feet, thighs, hips, buttocks, or, rarely, arms. Claudication is a manifestation of exercise-induced reversible ischemia, similar to angina pectoris. As PAD progresses, the distance that can be walked without symptoms may decrease, and patients with severe PAD may experience pain during rest, reflecting irreversible ischemia. Rest pain is usually worse distally, is aggravated by leg elevation (often causing pain at night), and lessens when the leg is below heart level. The pain may feel like burning, although this finding is nonspecific. About 20% of patients with PAD are asymptomatic, sometimes because they are not active enough to trigger leg ischemia. Some patients have atypical symptoms (e.g., nonspecific exercise intolerance, hip or other joint pain).

As ischemia worsens, ulcers may appear (typically on the toes or heel, occasionally on the leg or foot), especially after local trauma. The ulcers tend to be surrounded by black, necrotic tissue (dry gangrene). They are usually painful, but people with peripheral neuropathy due to diabetes or alcoholism may not feel them. Infection of ischemic ulcers (wet gangrene) occurs readily, producing rapidly progressive cellulitis.

The level of arterial occlusion influences location of symptoms. Aortoiliac PAD may cause buttock, thigh, or calf claudication; hip pain; and, in men, erectile dysfunction (Leriche syndrome). In femoropopliteal PAD, claudication typically occurs in the calf; pulses below the femoral artery are weak or absent. In PAD of more distal arteries, femoropopliteal pulses may be present, but foot pulses are absent.

Dependent on the severity of the disease, a spectrum of treatment options are available. Conservative measures include lifestyle changes such as smoking cessation (cigarettes promote PAD and are a risk factor for cardiovascular disease). Regular exercise for those with claudication helps open up alternative small vessels (collateral flow) and the limitation in walking often improves. Medication, which reduce clot formation and cholesterol levels, respectively can help with disease progression and address the other cardiovascular risks that the patient is likely to have. Angioplasty, cryotherapty, or stents (PTA or percutaneous transluminal angioplasty) can be done on lesions in all leg arteries. Plaque excision, in which the plaque is scraped, or undergoes laser removal, off of the inside of the vessel wall. Occasionally, bypass grafting is needed to circumvent a seriously stenosed area of the arterial vasculature. Generally, the saphenous vein is used, although artificial material is often used for large tracts when the veins are of lesser quality. Rarely, sympathectomy is used—removing the nerves that make arteries contract, effectively leading to vasodilatation. When gangrene of toes has set in, amputation is often a last resort to stop infected dying tissues from causing septicemia. Arterial thrombosis or embolism has a dismal prognosis, but is occasionally treated successfully with thromboylsis.

Surgery is indicated for patients who can safely tolerate a major vascular procedure and whose severe symptoms do not respond to noninvasive or catheter interventional treatments. The goal is to relieve symptoms, heal ulcers, and avoid amputation. Because many patients have underlying coronary artery disease, which places them at risk of acute coronary syndromes during surgical procedures for PAD, patients usually undergo cardiac evaluation prior to surgery. Thromboendarterectomy (surgical removal of an occlusive lesion) is used for short, localized lesions in the aortoiliac, common femoral, or deep femoral arteries. Revascularization (e.g., femoropopliteal bypass grafting) uses synthetic or natural materials (often the saphenous or another vein) to bypass occlusive lesions. Revascularization helps prevent limb amputation and relieve claudication. In patients who cannot undergo major vascular surgery, sympathectomy may be effective when a distal occlusion causes severe ischemic pain. Chemical sympathetic blocks are as effective surgical sympathectomy, so the latter is rarely done. Amputation is a procedure of last resort, indicated for uncontrolled infection, unrelenting rest pain, and progressive gangrene.

Routing a therapeutic catheter to a damaged artery can be difficult in patients suffering from PAD or other arterial diseases. Entering directly into an affected femoral artery, for instance routing a catheter from directly above the area and working a device straight down the affected femoral artery is quite difficult, requiring great skill on the part of the surgeon and significant time. Moreover there is a significant risk of further damaging already damaged arteries and complications approach 50%. A better method using therapeutic catheters is enter through a healthy artery that is distant from the damaged arteries to be treated. An alternative method would require entering through the arm and working a catheter downwards through the aorta and into the iliac artery and then into the affected femoral artery. This long route through the aorta can cause a great deal of discomfort for patients, and also entails relatively high complication rates, and frequently the therapeutic catheters are not long enough.

For example, in endovascular procedures to repair damage to a femoral artery damaged by PAD, the most preferred method would be to enter through a healthy femoral artery in the opposite leg from the procedure, advancing the catheter upwards into the iliac artery of the near leg (i.e. the leg not being repaired), make the U-turn through the lower aorta/common iliac artery and into the opposite side iliac artery, and then proceed down into the affected femoral artery for surgery. However, until now several problems can prevent this method from being used. First, therapeutic catheters are not flexible enough to make the bend from the near iliac artery into the opposite iliac artery and down into the femoral artery—referred to as the “U-turn”. This bend essentially entails a nearly 180 degree turn—impossible for currently available large-bore catheters. Second, flexible catheters, for example the SOS™ Omni™ catheter, can make the turn, but they cannot hold themselves in the opposite femoral artery when attempting to route a stiff guide wire through, which stiff guide wire could then be used to guide a therapeutic catheter. As a surgeon attempts to route a stiff guide wire through the flexible catheter, the flexible catheter displaces out of the femoral and iliac arteries when the stiff wire reaches the U-turn, and moves up into the aorta. At that point the stiff guide wire is not flexible enough to re-route the flexible catheter back into the iliac and femoral arteries. This same difficulty applies in other procedures where a therapeutic catheter must be routed to a location through a tortuous vascular path to support a stiff guide wire for therapeutic catheter routing. Therefore, until now PAD procedures on patients with difficult anatomy requiring entry from an opposite extremity could not be performed reliably, and the patients have been left with the option of no treatment or procedures with greater risks of complications.

To date most attempts at solving the problem have focused on increasing the flexibility of the tips of larger therapeutic catheters and stiff guide wires. This results in more expensive and complicated devices, as well as greater skill on the part of the surgeon to effectively use the devices. The difficulty of using the devices also increases the time required for a surgeon to complete a procedure, which both increases the costs and increases the potential for complications. The present invention seeks a solution through simplified components and a simple multi-step methodology that has not been used prior.

Therefore, a need exists for a method and apparatus which will allow a surgeon performing endovascular surgery, such as on a femoral artery, to anchor a flexible catheter in an upstream location in an artery so that a stiff guide wire can then be routed through the flexible catheter, which stiff guide wire can subsequently be used to route a medium or large catheter or sheath to conduct the actual therapeutic surgery, whether that involves an angioplasty balloon catheter, or inserting a stent, or some other sort of device or surgical procedure.

An explanation of terms is appropriate here. In the medical field catheters are widely used for many purposes, as are guide wires for such catheters. Certain terms of art have come to be understood in regards to catheters and guide wires in the field. Catheter sizes are described with a hodgepodge of units. They may be specified as having a specific outer diameter in inches or in millimeters, or using the French-scale where 3 french (“fr”) equals 1 mm—in this specification catheter diameters will be given in all three unit systems. Catheters may include one or more internal channels called lumens which are used for different functions, e.g. to accommodate a guide wire, to provide a channel for fluid to fill a balloon, to carry a fiber optic for a camera system or laser, etc. These channels may be arranged any number of ways: concentrically, eccentrically, parallel, or a combination of those, and the particular wall thickness will vary depending on the intended use of the lumen. A lumen intended to carry a gaseous fluid at high pressures for inflating a non-compliant balloon (such as angioplasty balloons to unblock an artery for instance) requires significantly greater wall thickness than a lumen for carrying saline to inflate a compliant balloon at low pressure. The walls of a gas lumen are also generally be made from stiffer material, thus a catheter including a gas lumen will have significantly less flexibility than needed for an anchor balloon catheter, for instance.

Lumens do not necessarily extend the entire length of a catheter, e.g. a lumen for inflating a balloon would only extend to the balloon port, but the catheter itself may extend beyond that point still including multiple lumens within the remaining length.

In this specification reference is made to an “SOS™ Omni™ catheter or “IMA™ catheter” which is used to insert an initial flexible or very-flexible guide wire. SOS™ Omni™ catheters and IMA™ catheters are a commercial lines of catheters including small diameter flexible catheters containing flexible or very-flexible guide wires in standard size ranges. Omni™ and IMA™ catheters are used herein to refer to flexible or very-flexible flow catheters, which are approximately 3 Fr (1 mm or 0.04 inches) outer diameter or less, with at least one wire lumen for accommodating a flexible or very-flexible guide wire.

“Flexible”, “very-flexible”, “stiff” and “very-stiff” in relation to guide wires and catheters have meanings generally understood in the medical fields. Guide wires for catheters are known in the art and made from materials such as polytetrafluoroethylene (PTFE) and similar biologically inert plastics, as well as coated metal. Such wires are generally categorized, in ascending order of rigidity, as: “very-flexible” (sometimes referred to as “glidewire” ), such as commonly used in SOS™ Omni™ catheters and IMA™ catheters; “flexible”; “moderately-stiff” (sometimes referred to as “house wire” or “general purpose wire”); “stiff”; “very-stiff”, and, “super-stiff.” Wire diameters typically range from 0.014 to 0.038 inches (0.34 mm to 0.97 mm), with 0.035 inches and 0.038 inches (0.89 mm and 0.97 mm) being most common. The thickness of a wire does not in itself determine its stiffness, however. For example, one may find flexible, stiff or super-stiff wires in the 0.035 inch diameter size. Medium and large-bore therapeutic catheters or sheaths (6-7 fr and greater (2 mm or 0.04 inches)) typically require stiff or super-stiff guide wires for support during routing, and therefore are too stiff to be guided through a tortuous vascular channel, such as past the U-turn of the common iliac artery in many patients. Based on these common conventions a person of ordinary skill in the art would be able to select an appropriate guide wire from the categories of very-flexible, flexible, stiff, very-stiff, or super-stiff. In the specification and claims, where reference is to a “flexible” wire, this includes wires that are more flexible as well, such as “very-flexible” wires. Where reference is made to “flexible” catheters, this includes catheters that are more flexible as well, such as “very-flexible” catheters.

The use of balloon catheters for therapeutic procedures is known. Therapeutic sheaths such as used for angioplasty, are relatively large requiring catheters of 7-8 Fr, and inflexible. The balloons are non-compliant, i.e. the balloon volume will remain constant as pressure is increased so that the balloon becomes stiff. This is a useful property for procedures such as angioplasty where the balloon is used to displace matter partially occluding an artery, or where a balloon must be used to place a stent. However, non-compliant balloons can easily damage weakened femoral arteries of patients suffering PAD, and non-compliant balloons require precise sizing and control to avoid inadvertently tearing smaller arteries apart and do not generally have adequate wire lumen size to accommodate a stiff wire. As such they are completely unsuitable for use as anchor balloons.

A common type of balloon catheter using a compliant balloon which allows a guide wire to go through it is a pulmonary artery catheter, referred to as a Swan-Ganz catheter. A compliant balloon is soft and flexible, so that it expands to maintain a stable low pressure when inflated (low meaning at or slightly greater than blood pressure). A Swan-Ganz catheter is generally a medium thickness (6-7 Fr), moderately-stiff, flow-directed catheter with a terminal inflatable balloon. It is inserted through the right atrium and ventricle into the pulmonary artery. The balloon is then inflated sufficiently to block the flow of blood from the right heart to the lung. So wedged, the catheter can provide a direct measurement of the “filling pressure” of the left ventricle of the heart. However, Swan-Ganz catheters are too large and inflexible to be used for the purposes of an anchor balloon catheter.

Currently, none of the procedures for treating PAD through the use of a catheter are able to cross from one extremity to the other through the arteries of a patient with difficult anatomy. Currently available balloon catheters are not useful for this purpose either. In this regard, “difficult anatomy” is the inability to pass a catheter from a proximal entry point to a location in an opposite side artery requiring a therapeutic procedure. This difficulty may be due, for example, to tortuous vessels with marked curvature, narrow iliac bifurcation with angles less than 45 degrees (i.e. a U-turn of nearly 180 degrees for femoral artery procedures), or due to previous endovascular intervention. None of the existing devices and procedures allow introduction into the femoral artery of the near leg of a patient with difficult anatomy, and progression of a therapeutic catheter to the femoral artery of the opposite leg.

Therefore, in order to get a catheter with therapeutic devices to a selected location in an artery for catheter based procedures to treat PAD, in patients with difficult anatomy, a surgeon must first pass a stiff wire from the near leg to the opposite leg via the common iliac artery and pass the therapeutic catheter over the stiff wire, or alternatively pass a sheath over the stiff wire through which a therapeutic catheter device may be passed. The problem therefore, is how to get such a stiff guide wire to the selected location. The present invention solves this problem.

The following represents a list of known related art:

| | | Date of |
|---|---|---|
| Patent/Publication | Name | Issue/Publication |
| US Pat 5,584,803 | Stevens et al. | Dec. 17, 1996 |
| US Pat 5,628,761 | Rizik | May 13, 1997 |
| US Pat 5,476,450 | Ruggio | Dec. 19, 1995 |
| US Pat 5,176,693 | Pannek, Jr. | Jan. 5, 1993 |
| US Pat 5,299,575 | Sandridge | Apr. 5, 1994 |
| US Pat 5,348,545 | Shani et al. | Sep. 20, 1994 |
| US Pat 5,456,665 | Postell et al. | Oct. 10, 1995 |
| US Pat 5,474,537 | Solar | Dec. 12, 1995 |
| US Pat 5,554,118 | Jang | Sep. 10, 1996 |
| US Pat 5,697,906 | Ariola et al. | Dec. 16, 1997 |
| US Pat 5,746,717 | Aigner | May 5, 1998 |
| US Pat 5,865,721 | Andrews et al. | Feb. 2, 1999 |
| US Pat 5,919,164 | Andersen | Jul. 6, 1999 |
| US Pat 6,129,708 | Enger | Oct. 10, 2000 |
| US Pat 6,602,270 B2 | Leschinsky et al. | Aug. 5, 2003 |
| US Pat 6,821,287 B1 | Jang | Nov. 23, 2004 |
| US Pat 7,066,872 B2 | Waksman et al. | Jun. 27, 2006 |
| US Pub 2002/0138093 A1 | Song et al. | Sep. 26, 2002 |
| US Pub 2002/0177867 A1 | Hebert et al. | Nov. 28, 2002 |
| US Pub 2003/0130610 A1 | Mager et al. | Jul. 10, 2003 |
| US Pub 2004/0019323 A1 | Carter et al. | Jan. 29, 2004 |
| US Pub 2004/0092868 A1 | Murray III | May 13, 2004 |
| US Pub 2004/0102734 A1 | Yozu et al. | May 27, 2004 |
| US Pub 2005/0065469 A1 | Tal | Mar. 24, 2005 |

The teachings of each of the above-listed citations (which does not itself incorporate essential material by reference) are herein incorporated by reference. None of the above inventions and patents, taken either singularly or in combination, is seen to describe the instant invention as claimed.

### SUMMARY AND ADVANTAGES

An anchor balloon for conducting peripheral vascular disease procedures in an opposite, or remote, extremity is provided, and includes a flexible catheter, which includes a wire lumen and a balloon lumen with a control port for connecting to balloon control and a balloon inflation port; and, a compliant anchor balloon connected near the flexible catheter distal end. A method for treating peripheral vascular disease in an opposite femoral artery includes inserting a first flexible catheter with flexible guide wire into an artery on an opposite side, advancing the flexible catheter to a location upstream of the surgery location, advancing the guide wire through the catheter to an anchor location, withdrawing the first catheter leaving the wire in place, inserting an anchor balloon catheter over the wire to an anchor location, withdrawing the first wire; inflating the anchor balloon against the arterial walls, inserting a stiff guide wire through the anchor balloon catheter to the point of surgery, deflating the anchor balloon, and withdrawing the anchor balloon catheter while leaving the stiff wire in place for further procedures.

The apparatus and methods of the present invention require significantly less surgical skill than routing a catheter from an arm or attempting a straight-on approach down the artery of the leg to be worked on. It is a more direct route than entering through an artery in the arm, but the entry point is sufficiently far from the point of the affected artery that less damage is risked than if a surgeon attempted an entry into the same side femoral artery—i.e. the damaged artery itself. The highly calibrated syringe of Carter (U.S. Pub 2004/0019323 A1) or other finely calibrated balloon inflation apparatus is not required in this procedure, but merely a basic catheter syringe. Due to the balloon being made from compliant material, and the fact that the inflated balloon diameter ranges only from 4-8 mm, the surgeon can simply inject saline into the balloon control port to expand the compliant balloon, tug lightly on the anchor balloon catheter to ensure it is set, and proceed to route the stiff wire. Expenses are thus saved, in the range of hundreds of dollars per anchor balloon catheter device, and a great deal of time is saved as well by the surgical team. This reduces costs, as well as the risk of complications—the longer any procedure takes the greater the risk of complications. The cost of an anchor balloon catheter is much less than the costs of multiple wires and catheters and can be used by less-skilled surgeons with less training required than existing apparatus and methods.

The apparatus and procedures disclosed here are also useful for procedures other than femoral artery surgery, where catheters with therapeutic devices must be routed through tortuous paths such as in the kidneys, liver, or other similar locations where it is difficult to route catheters and sheaths for therapeutic procedures. The main limitation would be procedures involving the carotid artery and beyond, due to the fact that even a momentary occlusion of the carotid artery by the anchor balloon could cause irreversible brain damage. A small diameter flexible or very flexible catheter flow catheter containing a flexible or very flexible guide wire may be used to route the guide wire to an anchor point. The initial catheter is withdrawn, an anchor balloon catheter is inserted over the flexible guide wire, the flexible guide wire is removed, the anchor balloon is inflated to hold position, a stiff or very-stiff guide wire is inserted through the anchor balloon catheter and advanced to the desired location, the anchor balloon is deflated, the anchor balloon catheter is removed over the stiff guide wire, and the stiff guide wire is then available for inserting a catheter or sheath for the therapeutic procedure.

The anchor balloon catheter and methods of the present invention therefore present numerous advantages, including: (1) allows use of therapeutic catheter procedures for treating PAD with entry from an opposite extremity in patients with difficult anatomy; (2) allows introduction into the femoral artery of one leg with difficult anatomy, and progression of a therapeutic catheter to the femoral artery of the opposite leg; (3) enables a surgeon to perform PAD catheter based surgery in difficult anatomy by enabling the surgeon to get a stiff guide wire to the arteries of the extremities; (4) is significantly less expensive and time consuming than current catheter procedures; and, (5) requires less skill and training for the surgeon; (6) is less likely to result in patient complications.

Additional advantages of the invention will be set forth in part in the description which follows, and in part will be obvious from the description, or may be learned by practice of the invention. The advantages of the invention may be realized and attained by means of the instrumentalities and combinations particularly pointed out in the appended claims. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows an embodiment of an anchor balloon catheter

FIG. 1A shows a cross-sectional view of an embodiment of an anchor catheter.

FIG. 1B shows a cross-sectional view of another embodiment of an anchor catheter.

FIG. 2 shows a view of an anchor balloon catheter focusing an the anchor balloon in place in a vessel with a cutaway.

FIG. 3 shows another view of an anchor balloon catheter with another cutaway.

FIGS. 4 -13 shows steps in a method for conducting peripheral vascular disease procedures using an anchored balloon catheter embodiment.

FIG. 4 shows placement of a short entry sheath in a near femoral artery.

FIG. 5 shows placement of an omni catheter with a flexible wire into the opposite common iliac artery.

FIG. 6 shows advancement of the flexible wire shown in FIG. 5 to the femoral artery of an opposing extremity.

FIG. 7 shows the flexible wire remaining in place after removal of the omni catheter shown in FIG. 6.

FIG. 8 shows placement of the novel anchor balloon catheter through the entry sheath, over the flexible wire to the femoral artery.

FIG. 9 shows the anchor balloon inflated to hold position in the femoral artery after the flexible wire is removed and awaiting insertion of the stiff wire.

FIG. 10 shows a stiff wire advanced through the anchor balloon catheter past the inflated anchor balloon to a location in the lower femoral artery.

FIG. 11 shows the stiff wire left in place with the short entry sheath after withdrawal of the anchor balloon catheter.

FIG. 12 shows the stiff wire left in place after withdrawal of the short entry sheath.

FIG. 13 shows a long large diameter sheath advanced into the common iliac artery over the stiff wire.

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

An anchor balloon catheter for conducting peripheral vascular disease procedures in an opposite, or remote, extremity is provided, and includes a flexible catheter, which includes a wire lumen and a balloon lumen with a control port for connecting to balloon control and a balloon inflation port; and, a compliant anchor balloon connected near the flexible catheter distal end. A method for treating peripheral vascular disease in an opposite femoral artery includes inserting a first flexible catheter with flexible guide wire into an artery on an opposite side, advancing the flexible catheter to a location upstream of the surgery location, advancing the guide wire through the catheter to an anchor location, withdrawing the first catheter leaving the wire in place, inserting an anchor balloon catheter over the wire to an anchor location, withdrawing the first wire; inflating the anchor balloon against the arterial walls, inserting a stiff guide wire through the anchor balloon catheter to the point of surgery, deflating the anchor balloon, and withdrawing the anchor balloon catheter while leaving the stiff wire in place for further procedures.

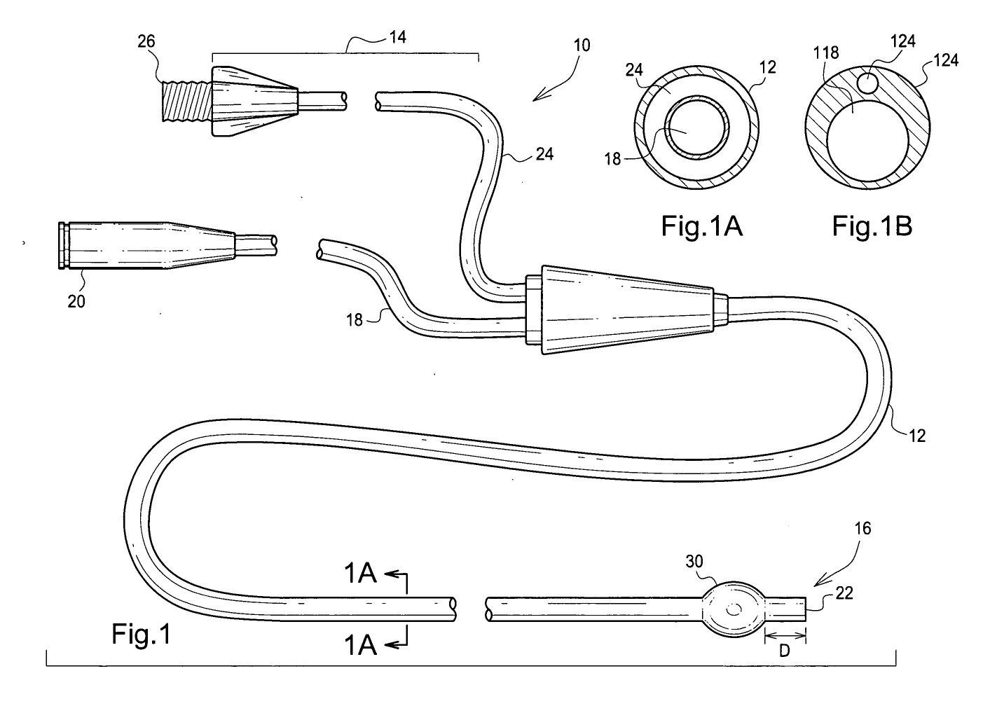

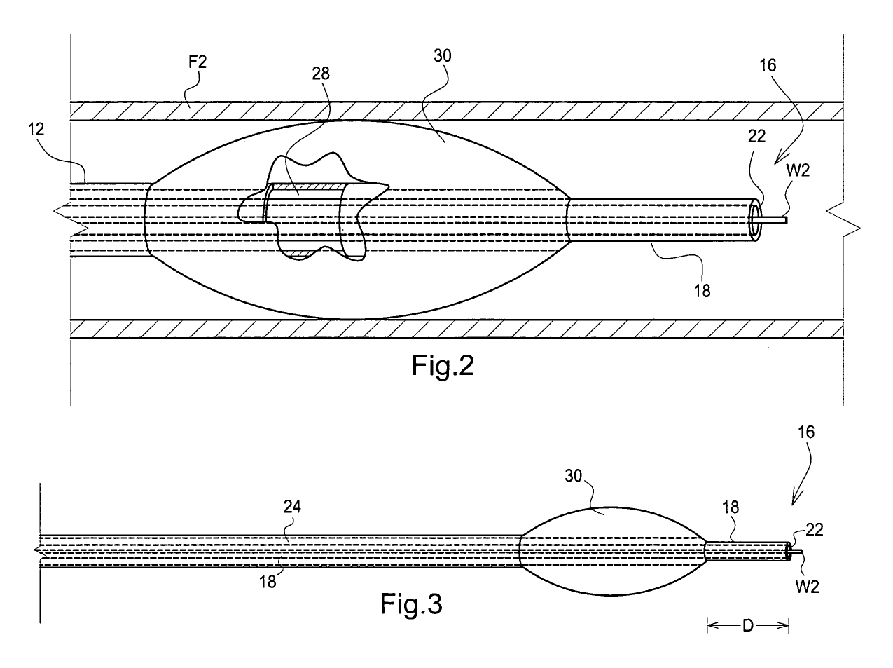

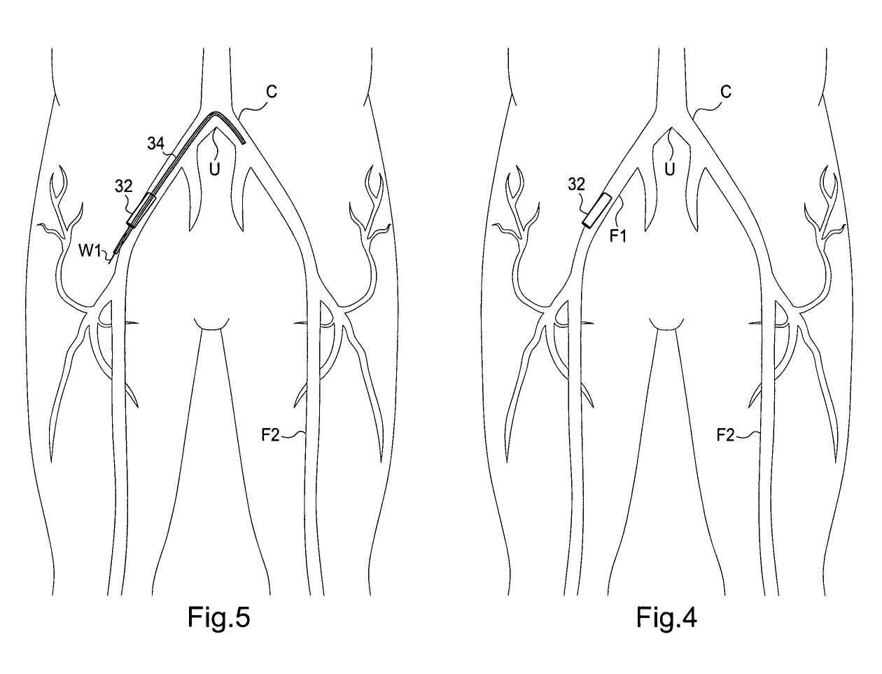

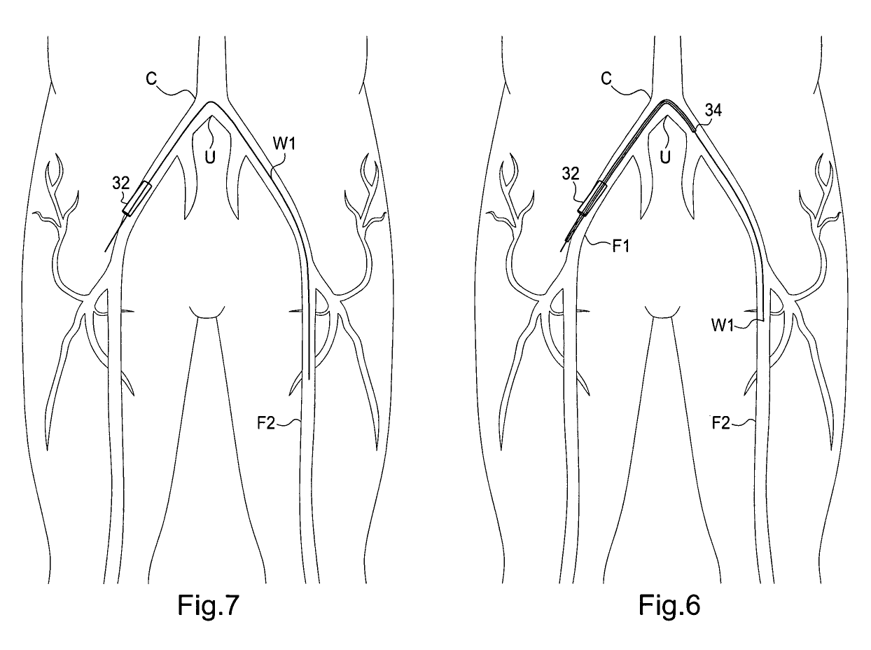

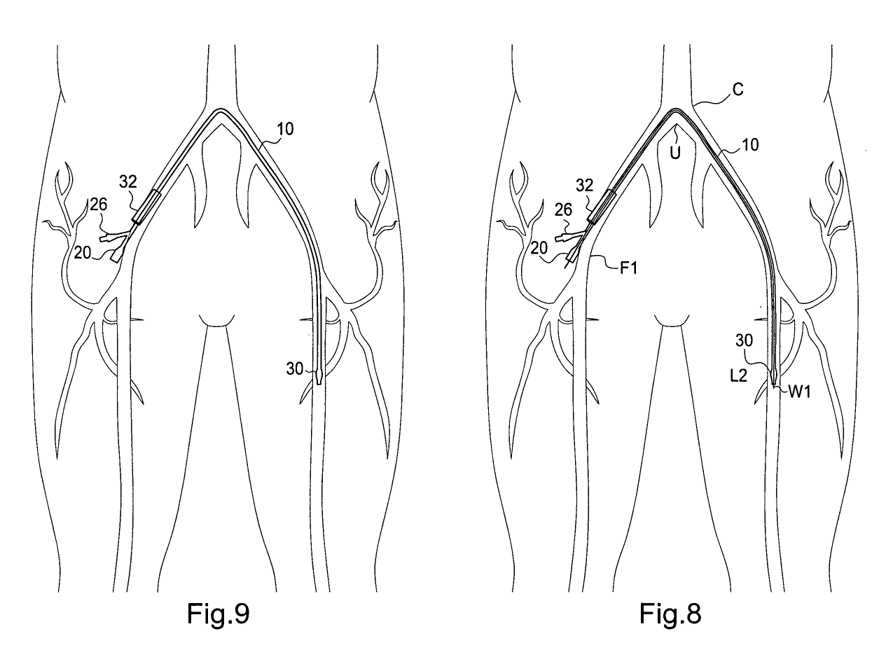

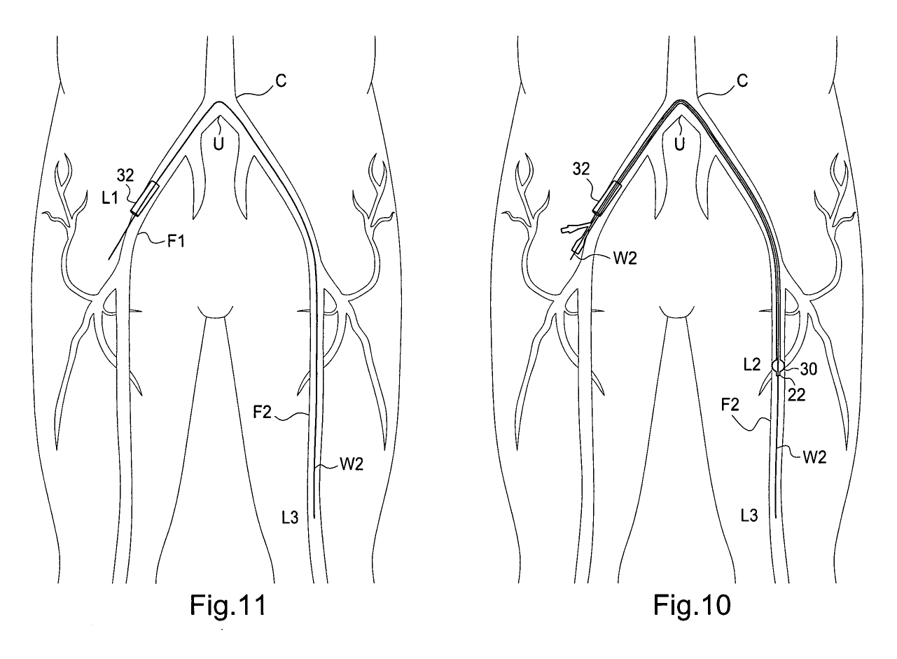

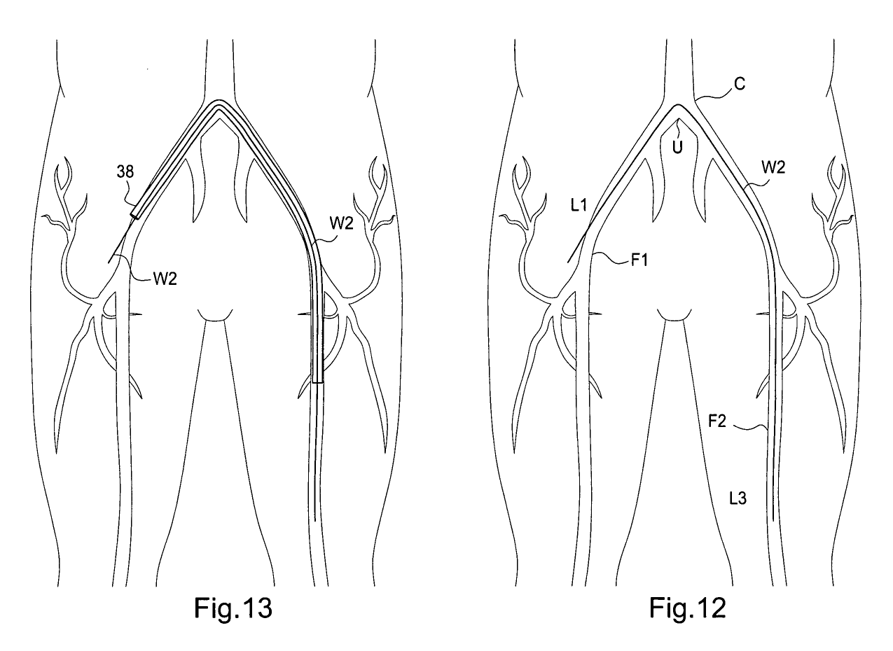

In a preferred embodiment of an anchor balloon catheter for conducting vascular procedures from a remote entry point, such as an opposite extremity, shown in FIGS. 1-3, and referring to FIGS. 4-14, an anchor balloon catheter 10 includes a flexible catheter 12 including proximal end 14 and distal end 16, and a plurality of lumens 18 and 24, with flexible catheter 12 having length sufficient to reach a selected anchor location L2 in a patient's opposite iliac artery; wherein the plurality of lumens comprises at least a guide wire lumen 18 including a wire access port 20 accessible to a surgeon at the flexible catheter proximal end 14 and a wire exit port 22 at the flexible catheter distal end 16; and, a balloon lumen 24, said balloon lumen 24 including a balloon control port 26 for connecting to inflation control means (not shown) accessible to a surgeon at the flexible catheter proximal end 14 and a balloon inflation port 28 near flexible catheter distal end 16; and, a compliant anchor balloon 30 connected to flexible catheter 12 at a selected distance D from said flexible catheter distal end 16 and in fluid communication with said anchor balloon control port 20 via said balloon operating port 28 and balloon lumen 24.

In an embodiment, flexible catheter 12 preferably has an outer diameter less than or equal to approximately 3 Fr (1 mm or 0.039 inches), and preferably has a length of approximately 90 cm (35.4 inches) in order to reach from an entry point L1 in a near femoral artery to an anchor point L2 in the opposite iliac artery through entry sheath 32.

As shown in FIGS. 1-3, anchor balloon 30 is preferably connected a distance D of approximately 10 mm (0.4 inches) to 20 mm (0.8 inches) from the distal end 16 of flexible catheter 12 to maintain adequate flexibility for routing anchor balloon catheter 10. Anchor balloon 30 is preferably approximately 10 mm (0.4 inches) in length and has a deflated diameter of less than 6 fr (2 mm or 0.08 inches), so it may pass through entry sheath 32 and not occlude the arteries when deflated, and has an inflated diameter of approximately 4 mm (0.16 inches) to approximately 8 mm (0.31 inches), so that it will definitely contact the arterial walls and occlude the artery to hold its position when inflated at a selected anchor location. Arterial occlusion assists anchor balloon 30 in holding position due to the effect of blood pressure added to the contact with the arterial wall. An inflated range of 4 mm to 8 mm is wide enough to ensure the ability to anchor in most situations where endovascular procedures are contemplated. However, an anchor balloon 30 with inflated diameter greater than 8 mm (0.31 inches) may be required in specific cases and is contemplated as within the scope of the invention.

Anchor balloon 30 is inflated via balloon lumen 24 which is in fluid communication with balloon 30 through balloon inflation port 28 and in fluid communication with balloon inflation means (not shown) through balloon control port 26. Anchor balloon 30 is preferably inflated using saline or other surgically compatible fluid. Anchor balloon 30 is made from compliant flexible material, such as latex or similar compliant medically-compatible materials, so that it maintains an essentially constant pressure throughout its inflated diameter range. The diameter of anchor balloon 30 when fully inflated is such as to occlude the vascular channel at the selected anchor location, in this example a point past the U-turn of the common iliac artery C and within the opposite side iliac artery (for femoral artery procedures), although other locations may be used depending on the specific surgery to be performed. In the present embodiment balloon lumen 24 is concentric with both flexible catheter 12 and wire lumen 18. As shown in FIG. 1B, wire lumen 118 and balloon lumen 124 may be arranged parallel but nonconcentric as well.

Various balloon inflation means are known and described in the art and are compatible with the presently described apparatus and method. Syringes containing saline are commonly used. Balloon lumen 24 is sealed at its distal end while wire lumen 18 is open to allow wires W1 and W2 to pass through. The distance D from the distal end of balloon 30 to the distal end 16 of the flexible catheter 12 is approximately 1 to 2 cm (0.4 to 0.8 inches). This distance allows the distal end of flexible catheter 12 to remain as flexible as possible in order to be threaded over flexible guide wire W1 through tortuous channels without causing flexible guide wire W1 to displace.

Wire lumen inside diameter 18 is less than 3 Fr (1 mm or 0.04 inches) and preferably has an inside diameter of approximately 0.91 mm (0.036 inches) to accommodate a thin 0.035 inch flexible guide wire W1, as flexible 0.035 inch guide wires are universally available and surgeons are very familiar with the classifications and characteristics of flexible and stiff 0.035 inch guide wires. Additionally, manufacturers are familiar with techniques for producing flexible catheters with wire lumens to accommodate a range of 0.035 inch guide wires, so they may be produced at relatively low cost. However, guide wires and corresponding wire lumens of other diameters are contemplated within the scope of this invention, so long as the guide wire W1 and catheter 12 are classified as at least “flexible” or “very flexible”, as defined above. Preferably flexible guide wire W1 is a glidewire as commonly used in SOS™ Omni™ or IMA™ catheters, made from PTFE or other hydrophobic material to make passage through the arteries easier. As shown in FIGS. 1-3, in a preferred embodiment balloon lumen 24 is formed by inserting the tube of wire lumen 18 into a sheath which is sealed around the outer diameter of wire lumen 18. Although narrow, balloon lumen 24 is sufficient to transmit low viscosity fluid such as saline to balloon 30. An alternative arrangement is shown in FIG. 14, where a small parallel channel is provided for balloon lumen 24. Other arrangements of internal lumen are known and within the skill of catheter makers.

In operation, an anchor balloon catheter as disclosed is used in an intermediate step of the disclosed surgical methods. Anchor balloon catheter 10 is a device meant to give access to place a therapeutic delivery system for the treatment of peripheral vascular disease or peripheral artery disease (PAD). As shown in FIGS. 4-13, and FIGS. 1-3, a method for performing femoral vascular surgery includes the steps of: inserting a short entry sheath 32 into an entry point L1 in a near femoral artery F1 (see FIG. 4); inserting a first flexible catheter 34 including a flexible guide wire W1 through said entry sheath 32 and advancing said first flexible catheter 34 past the U-turn of the common iliac artery U into the opposite channel of the common iliac artery C (see FIG. 5); advancing said flexible guide wire W1 through said first flexible catheter 34 into said opposite femoral artery F2; withdrawing said first flexible catheter 34 through said entry sheath 32 over said flexible guide wire W1 while leaving said flexible guide wire W1 in place; inserting an anchor balloon catheter 10 through said entry sheath 32 and over said flexible guide wire 34 using said wire lumen 18 of said anchor balloon catheter 10, and advancing said anchor balloon catheter 10 along said flexible guide wire W1 until said anchor balloon 30 is located at said anchor location L2; withdrawing said flexible guide wire W1 through said anchor balloon catheter 10 and said entry sheath 32; inflating said anchor balloon 30 against the arterial walls at said anchor location L2; inserting a stiff guide wire W2 through a wire lumen 18 within said anchor balloon catheter 10 and advancing said stiff guide wire W2 past said inflated anchor balloon 30 to the selected location for the vascular femoral surgery L3; deflating said anchor balloon 30; and, withdrawing said anchor balloon catheter 10 through said entry sheath 32 over said stiff guide wire W2 while leaving said stiff guide wire W2 in place; withdrawing entry sheath 32 over said stiff guide wire W2 leaving stiff guide wire W2 in place; wherein said stiff guide wire W2 is then available to insert a long sheath 38 or therapeutic catheter for therapeutic procedures.

Entry sheath 32 is preferably a short sheath of 6 Fr or greater inner diameter (2 mm or 0.08 inches). Entry sheath 32 may be used to protect the femoral artery from damage when inserting and removing catheters during the procedure, or for inserting other devices in the course of the procedure in parallel with the anchor balloon catheter 10.

Flexible guide wire W1 and stiff guide wire W2 are preferably no greater than 0.91 mm (0.035 inches) in diameter so that anchor balloon catheter 10 with internal wire lumen 28 may be limited to 3 Fr or less in diameter (1 mm or 0.039 inches). Flexible guide wire W1, as described above, has minimum bend radius sufficient to be directed around the particular patient's common iliac artery U-turn U, or other specific obstruction as determined by the surgeon. Stiff guide wire W2 must have sufficient stiffness to be pushed through wire lumen 28 in anchor balloon catheter 10, and to support the insertion and advancement of a large diameter sheath 36 after withdrawal of anchor balloon catheter 10. Large diameter sheath 36 is typically 6-7 Fr or greater in diameter to accommodate therapeutic catheters or other devices for surgery. Large diameter sheath 36 is preferably at least 45 cm (17.7 inches), or at least long enough to advance into the opposite channel of the particular patient's common iliac artery

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

1. An anchor balloon catheter for conducting vascular procedures upon arteries in an extremity from an opposite entry point, comprising:
a flexible catheter including a proximal end and a distal end, and a plurality of lumens, said flexible catheter having length sufficient to reach a selected location in a patient's opposite extremity, wherein said plurality of lumens comprises at least:
a wire lumen including a wire access port accessible to a surgeon at said flexible catheter proximal end and a wire exit port at said flexible catheter distal end, and
a balloon lumen, said balloon lumen including a balloon control port for connecting to balloon control means accessible to a surgeon at said flexible catheter proximal end and a balloon inflation port near said flexible catheter distal end; and,
a compliant anchor balloon connected to said flexible catheter at a selected distance from said flexible catheter distal end and in fluid communication with said anchor balloon control port via said anchor balloon operating port.

2. The apparatus of claim 1, wherein said flexible catheter outer diameter is less than or equal to approximately 1 mm (0.039 inches).

3. The apparatus of claim 1, wherein said flexible catheter is approximately 90 cm (35.4 inches) in length.

4. The apparatus of claim 1, wherein distance of the distal end of said anchor balloon from the distal end of said flexible catheter lies in the range 10 mm (0.4 inches) to 20 mm (0.8 inches).

5. The apparatus of claim 1, wherein said anchor balloon is approximately 10 mm (0.4 inches) in length and has a deflated diameter of approximately 4 mm (0.2 inches) and an inflated diameter of approximately 8 mm (0.3 inches).

6. The apparatus of claim 1, wherein the balloon diameter when fully inflated is such as to occlude the vascular channel at said selected location.

7. The apparatus of claim 1, wherein the wire lumen diameter is less than or equal to 0.91 mm (0.035 inches).

8. An anchor balloon catheter, comprising:
a flexible catheter insertable into a femoral artery having a port end and a distal end, wherein said port end is provided with a plurality of ports and wherein said catheter is provided with a plurality of lumens corresponding to said plurality of ports;
inflatable compliant stabilizing means provided near said distal end of said flexible catheter, in fluid communication with one of said lumens and one of said ports, which occludes the artery when inflated and thereby stabilizes said flexible catheter to allow a stiff guide wire to pass through said flexible catheter from one extremity to the opposite extremity.

9. A method for treating peripheral vascular disease in an opposite femoral artery, comprising the steps of:
inserting a short entry sheath into an entry point in a near femoral artery;
inserting a first flexible catheter including a flexible guide wire through said sheath and advancing said omni catheter past the U-turn of the common iliac artery to a location in the opposite channel of the common iliac artery;
advancing said flexible wire through said omni catheter into said opposite femoral artery;
withdrawing said first flexible catheter through said sheath over said flexible guide wire while leaving said flexible guide wire in place;
inserting an anchor balloon catheter through said sheath and over said flexible guide wire using said wire lumen of said anchor balloon catheter, and advancing said anchor balloon catheter along said flexible guide wire until said anchor balloon is located at said anchor location;
withdrawing said flexible guide wire through said anchor balloon catheter and said entry sheath;
inflating said anchor balloon against the arterial walls at said anchor location;
inserting a stiff guide wire through a wire lumen within said anchor balloon catheter and advancing said stiff wire past said inflated anchor balloon to the selected location for the vascular femoral surgery;
deflating said anchor balloon;
withdrawing said anchor balloon catheter through said sheath over said stiff wire while leaving said stiff wire in place; and,
withdrawing said entry sheath over said stiff wire while leaving said stiff wire in place;
wherein said stiff wire is then available to insert a long sheath or therapeutic catheter for therapeutic procedures.

10. The method of claim 9, wherein said flexible guide wire is less than or equal to 0.035 inches (0.91 mm) in diameter.

11. The method of claim 9, wherein said stiff guide wire is less than or equal to 0.035 inches (0.91 mm) in diameter.

12. A method for performing vascular surgery on a patient, comprising the steps of:
inserting a first flexible catheter including a flexible guide wire through into an entry point in an artery on the opposite side of said patient;
advancing said first flexible catheter through the arteries of said patient to a location upstream of the intended location of said vascular surgery;
advancing said flexible guide wire through said first flexible catheter to an anchor location;
withdrawing said first flexible catheter over said flexible guide wire while leaving said flexible guide wire in place;
inserting an anchor balloon catheter over said flexible guide wire using said wire lumen of said anchor balloon catheter, and advancing said anchor balloon catheter along said flexible guide wire until said anchor balloon is located at said anchor location;
withdrawing said flexible guide wire through said anchor balloon catheter;
inflating said anchor balloon against the arterial walls at said anchor location;
inserting a stiff guide wire through a wire lumen within said anchor balloon catheter and advancing said stiff wire past said inflated anchor balloon to the intended location for the vascular surgery;
deflating said anchor balloon;
withdrawing said anchor balloon catheter over said stiff wire while leaving said stiff wire in place;
wherein said stiff wire is then available to insert a long sheath or therapeutic catheter for therapeutic procedures.
